(19) United States
(12) Patent Application Publication
(10) Pub. No.: US 2022/0033752 A1
(43) Pub. Date: Feb. 3, 2022

(54) BIOREACTOR FOR CELL CULTURE

(71) Applicants: Prakashkumar Narasimhamurthy, London (GB); Yougunn Prakashkumar, London (GB); Saradha Prakashkumar, London (GB)

(72) Inventors: Prakashkumar Narasimhamurthy, London (GB); Yougunn Prakashkumar, London (GB); Saradha Prakashkumar, London (GB)

(73) Assignee: Prakashkumar Narasimhamurthy, London (GB)

(21) Appl. No.: 17/100,972

(22) Filed: Nov. 23, 2020

(30) Foreign Application Priority Data
Jul. 31, 2020 (GB) 2011948.3
Sep. 21, 2020 (EP) PCT/EP2020/076314

(51) Int. Cl. C12M 1/12; C12M 1/00; C12M 3/00

(52) U.S. Cl. CPC C12M 25/02; C12M 23/34; C12M 23/42; C12M 27/20; C12M 25/14; C12M 25/18; C12M 23/44

(57) ABSTRACT

A bioreactor for cell culture comprising, a first set of open top troughs that are interconnected by a first set off low channels to form a first cascade, a second set of open top troughs that are interconnected by a second set of flow channels to form a second cascade, and a set of interconnecting flow channels coupled between the first cascade and the second cascade, wherein the first cascade circulates a fresh media and the second cascade circulates a cell culture and a first quantity of the fresh media from first cascade is supplied to the second cascade through the set of interconnecting flow channels when a second quantity of cell culture is removed from the second cascade.

### CROSS REFERENCES TO RELATED APPLICATIONS

This application claims priority from GB patent application No.: 2011948.3 filed on Jul. 31, 2020 and International application No.: PCT/EP2020/076314 Filed on Sep. 21, 2020 that are incorporated herein in its entirety by reference.

### BACKGROUND

### Technical Field

Embodiments of the present disclosure relates generally to a bioreactor and more specifically to a production scale bioreactor providing efficient gas exchange to a cell culture with reduced hydrodynamic stress placed upon cells, mimicking the in vivo environment in vitro thereof.

### RELATED ART

A bioreactor is typically a vessel provided with a provision of cell cultivation under sterile and controlled environmental conditions such as pH, temperature, dissolved oxygen, gases, and the like. Conventional bioreactor comprises a jacket or some similar means to transfer heat, a sparger to provide oxygen, and an agitator to mix the contents in the bioreactor which helps in transportation of gases and nutrients to the cell culture. However, bubbling and stirring actions caused by the sparger and agitators exert hydrodynamic stresses to the growing cells resulting in their high cell mortality rates.

The high cell mortality rate also occurs due to the increase in the cell culture volume as it leads to inefficient gas exchange despite the application of bubbling and stirring actions to the cell culture in the bioreactor. In order to increase the volume of the cell culture in a bioreactor, the height and perimeter or the diameter of the bioreactor needs to be increased which poses difficulty in maintaining optimum gas exchange.

The waste gases, i.e. carbon dioxide, released by millions of cells within the bioreactor must be removed and continuously replaced with the fresh gases, i.e. oxygen, required for growth. If this gas exchange is not efficient, the cells are more likely to die as a result of being surrounded by their own metabolic waste gases, therefore becoming starved of oxygen. This may also lead to mass death of the growing cells. It is very difficult to accurately ascertain when such mass death may occur during the cell culture process.

As cells grow heavier, they will sink due to gravity. The cell population increases and becomes congested at the bottom of the bioreactor. It becomes increasingly hard to supply nutrients and the fresh gases to this growing congestion of cell population. The stirring and bubbling actions together spread the congestion of cells evenly throughout the culture volume. The bubbling delivers the fresh gas required for cell intake and removes the metabolic waste gases from the cell culture. However, this method is proved to be inefficient for gas exchange as the increased intensity of bubbling and stirring naturally raises the mortality rates due to hydrodynamic stress. This problem becomes intensified with the increasing height of the cell culture volume contained in a bioreactor. Also, the technological limitations to cool the entire bulk volume of cell culture contained in a bioreactor when needed (for example in a jacket heated bioreactor; are higher when the volume of cell culture in the bioreactor increases.

Higher mortality rates and the inefficient removal of the metabolic waste gases turn the cell culture into a toxic mixture which needs to be discarded and replenished with a fresh nutrient media. However, it is to be noted that the replenished cell culture is still toxic to the cells and the only solace being that it is less toxic before becoming more toxic. Also, there is always a probability of removing from a continuously stirred and bubbled cell culture a larger quantity of delicate immature cells before they grow into robust mature cells. When such removed cell culture is sent to a Cell Retention Device, in a perfusion process, immature cells maybe lost due to mortality and clogging in the filter.

Stirring and bubbling should be paused during a process to allow gravity settling of cells to the bottom of the bioreactor. Alternatively, the continuously stirred and bubbled cell culture is pumped out of the bioreactor into a gravity settler where the cells are separated from the supernatant. This enables removal of a predetermined quantity of clearer supernatant from near the surface of the cell culture in the bioreactor or the gravity settler. The removed supernatant is discarded and replaced by supplying fresh nutrient into the bioreactor, and the gravity settled cells remain in the bottom of the bioreactor or is transferred from the gravity settler back to the bioreactor. When stirring and bubbling is paused in the methods above, the cells congest at the bottom and are deprived of oxygen and nutrients, therefore can be detrimental for the growing cells.

There is always a possibility that contaminants may reach the cell culture inside the bioreactor through leaks in bioreactor body which increases the risk of cell mortality.

Scientists involved in cell culture experimentation face several challenges. These include maintaining oxygen concentrations for culture, which if insufficient can be detrimental for culture viability and reproducibility. This poses difficulties for breakthrough cell culture models to progress into cures for human disease. Furthermore, these challenges are greater when growing larger volumes of cell culture in a bioreactor.

Maintaining consistent oxygen concentrations with reduced hydrodynamic stresses placed upon cells in vitro, that mimics an in vivo environment, will greatly enhance studies in cell research. This is further augmented if done in a production scale bioreactor. This would overall lead to the successful development and the production of innovative treatments to counter diseases and pandemics.

### SUMMARY

According to an aspect of the present disclosure, a bioreactor for cell culture comprising, a first set of open top troughs that are interconnected by a first set of flow channels to form a first cascade, a second set of open top troughs that are interconnected by a second set of flow channels to form a second cascade, and a set of interconnecting flow channels coupled between the first cascade and the second cascade, wherein the first cascade circulates a fresh media and the second cascade circulates a cell culture and a first quantity of the fresh media from first cascade is supplied to the second cascade through the set of interconnecting flow channels when a second quantity of cell culture is removed from the second cascade.

According to another aspect of the present disclosure, the first quantity received into the bioreactor and into the first cascade being proportional to the second quantity removed from the second cascade and out of the bioreactor.

Several aspects are described below, with reference to diagrams. It should be understood that numerous specific details, relationships, and methods are set forth to provide a full understanding of the present disclosure. One who is skilled in the relevant art, however, will readily recognize that the present disclosure can be practiced without one or more of the specific details, or with other methods, etc. In other instances, well-known structures or operations are not shown in detail to avoid obscuring the features of the present disclosure.

### BRIEF DESCRIPTION OF DRAWINGS

FIG. 1 is a diagram illustrating a bioreactor providing gas exchange to a cell-culture with reduced hydrodynamic stress in an embodiment of the present disclosure.

FIG. 2 is a diagram illustrating a bioreactor with different loops of fresh media and cell culture for an efficient gas exchange to the cell culture in another embodiment of the present disclosure.

FIG. 3 is a diagram illustrating a bioreactor with plurality of troughs in a single loop providing gas exchange to a cell-culture with reduced hydrodynamic stress in another embodiment of the present disclosure.

FIG. 4 is a diagram illustrating a bioreactor with plurality of troughs in different loops providing continuous yield with reduced hydrodynamic stress in yet another embodiment of the present disclosure.

FIG. 5 is a diagram illustrating a bioreactor comprising at least two cascades accelerating the yield of cells with reduced hydrodynamic stress in yet another additional embodiment of the present disclosure.

### DETAILED DESCRIPTION OF THE PREFERRED EXAMPLES

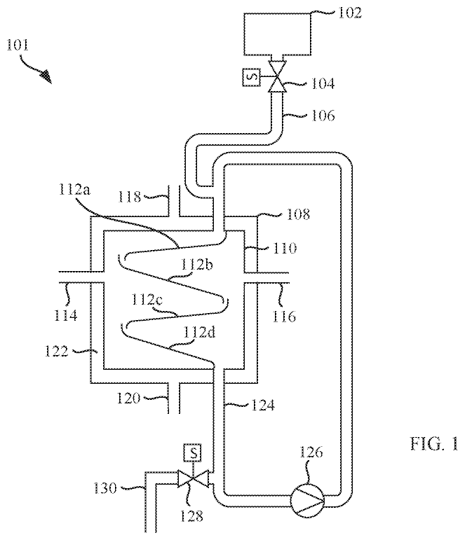

FIG. 1 is a diagram illustrating a bioreactor (101) providing efficient gas exchange to a cell-culture in the absence of stirring and bubbling to the cell culture with reduced hydrodynamic stress placed upon cells in an embodiment of the present disclosure. The bioreactor (101) comprises a reservoir (102), an outer compartment or container (108), an inner compartment or container (110), at least one channel (112a through 112d) and a pump (126). Each element is further described as follows.

The reservoir (102) is configured to source or feed a stock cell culture onto the channel (112a) through an inlet pipe (106) at a regulated flow rate. The flow rate of the stock cell culture is controlled by operating a first valve (104) connected to the reservoir (102). In an embodiment, the reservoir is either manually fed or automatically filled with the seed cell culture mixed with the fresh media or the cell culture by an external device or apparatus wherein the reservoir (102) holds the stock to a quantity of predetermined value.

The outer compartment (108) comprises a first inlet (118) and a first outlet (120) for the passage of a sterilising gas. The inner compartment (110) resides within the outer compartment (108) leaving a passage (122) between inner contour of the outer compartment (108) and outer contour of the inner compartment (110) facilitating free flow of the sterilising gas. Thus, forming a sterile envelop around the inner compartment (thus providing a bioreactor with a sterile envelop). In one embodiment, the inlet pipe (106) and an outlet pipe (124) are connected to the inner compartment (110) through the outer compartment (108) for introducing and removing the cell culture from the flow channels (112a through 112d).

In an embodiment, the inner compartment comprises a second inlet (114) and a second outlet (116) passing through the outer compartment (108) which helps in exchange of gases. The second inlet (114) allows the passage of fresh gas into the inner compartment (110) while the waste gases from the inner compartment i.e., the waste metabolic gases released during cell cultivation are allowed to leave through the outlet (116).

The inner compartment (110) is configured to hold the flow channels (112a through 112d) in that, the flow channels (112a through 112d) receive the seed cell culture mixed with fresh media or cell culture from the reservoir (102) through the inlet pipe (106) that is passing through the outer compartment (108). In an embodiment, the flow channels (112a through 112d) are inclined at the bottom and are positioned in such a way that it substantially exposes the cell culture on the channel to a stream of fresh gas entering through the second inlet (114) of the inner compartment (110). The inclination of the channels (112a through 112d) at the bottom allows the cell culture from the inlet pipe to flow gently downwards on the surface of the flow channels (112a through 112d) due to gravity.

In another embodiment, the plurality of flow channels (112a through 112d) are placed one above the other forming a stack maintaining equal gaps between each flow channel so that each channel (112a through 112d) connect the cell culture flow between the inlet and outlet pipes (106 and 124). Further, each flow channel (112a through 112d) comprises a plurality of passages substantially exposing the bottom and sides of the cell culture stream on the channel (112a through 112d) to the fresh gas. The cell culture flowing in the flow channels (112a through 112d) is thus exposed to the fresh gases and flows outside through the outlet pipe (124) connected to the inner compartment (110). In an example, the inlet pipe (106) is always positioned above the outlet pipe (124) for free flow of cell culture. The obtained cell culture at the outlet pipe (124) is again re-circulated back to the inlet pipe (106) either before or after externally harvesting the cells from the cell culture by using a connector pump (126). The cultured cells may be removed from the outlet pipe (124) through an external pipe (130) by operating a second valve (128). Thus, exchange of gases to the cell-culture takes place in the absence of stirring and bubbling actions and reduces hydrodynamic stress thereby reducing the cell mortality rate.

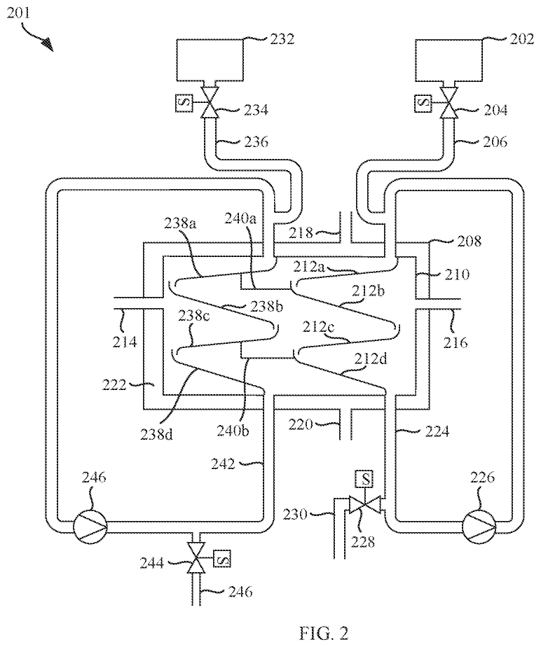

FIG. 2 is a diagram illustrating a bioreactor (201) with different loops of fresh media and cell culture for an efficient gas exchange to the cell culture in the absence of stirring and bubbling to the cell culture with reduced hydrodynamic stress placed upon cells in another embodiment of the present disclosure. As shown there, the bioreactor (201) comprises a first reservoir (202), a second reservoir (232), a first set of flow channels (212a through 212d), a second set of flow channels (238a through 238d), a plurality of connecting means (240a and 240b), a first inlet and outlet pipes (206 and 224), a second inlet and outlet pipes (236 and 242), an outer compartment (208) and an inner compartment (210). In one embodiment, the bioreactor comprises two different loops in that a first loop allows a seed cell culture mixed fresh media or cell culture to flow from the first reservoir (202) to the first outlet pipe (224) through the first inlet pipe (206) and the first set of flow channels (212a through 212d) while the second loop allows the flow of a fresh media from the second reservoir (232) to the second outlet pipe (242) through the second inlet pipe (236) and the second set of flow channels (238a through 238d).

In an embodiment, the first reservoir (202), the first inlet pipe (206), a first valve (204), the first set of flow channels (212a through 212d), the first outlet pipe (224) and the first connector pump (226) are coupled to each other and are functional in a similar way to that of the bioreactor (101) as discussed in the FIG. 1. Also, the outer and inner compartments (208 and 210) comprises an inlet and outlet (218 and 220) to the outer compartment (208) for the introducing and removing a sterilising gas through the passage (222) formed between the outer and inner compartments (208 and 210). Further, the inner compartment (210) is provided with an inlet and outlet pipes (214 and 216) for the passage of fresh gases into the compartment (210) and removal of waste gases from the compartment (210). The first inlet and outlet pipes (206 and 224) facilitate the introduction and removal of cell culture from the first set of channels (212a through 212d) within the inner compartment (210). The cell culture from the outlet pipe (224) may be removed from an external pipe (230) by operating a second valve (228) or else resend it to the first inlet pipe (206) by a first connector pump (226) either before or after externally harvesting the cells.

In another embodiment, the second reservoir (232), second inlet pipe (236), second valve (234), the second set of flow channels (238a through 238d), the second outlet pipe (242) and the second connector pump (246) are operated or functional within the inner compartment in such a way similar to that of the elements as discussed in the bioreactor (101) of the FIG. 1. The second inlet and outlet pipes (236 and 242) facilitate the introduction and removal of a fresh media from the second set of flow channels (238a through 238d) within the inner compartment (210). The fresh media overflowing from the second set of channels (238a through 238d) through second outlet pipe (242) may be removed from an external pipe (246) by operating a third valve (244) or else resend it to the second inlet pipe (236) by a second connector pump (246).

In yet another embodiment, the first set of flow channels (212a through 212d) and the second set of flow channels (238a through 238d) are interconnected to each other by a plurality of connecting means (240a and 240b) that facilitate the flow of fresh media from second set of flow channel (238a) to the first set of flow channel (212b) where the cell culture utilises the nutrients from the fresh media for their growth. In one embodiment, the connecting means may comprise at least one connecting pipe, permeable or semi-permeable membrane and the like that are coupled to the second set of flow channels (238a) and the first set of flow channels (212b). Further, the connecting means (240a) connects the second set of channels (238a) positioned at a higher level to that of the first set of channels (212b). the fresh air or gases from the inlet pipe (214) enters the inner compartment (210) wherein the fresh media on the second set of channels (238a through 238d) as well as cell culture on the first set of channels (212a through 212d) are exposed to the fresh gases while waste gases are removed from the outlet pipe (216).

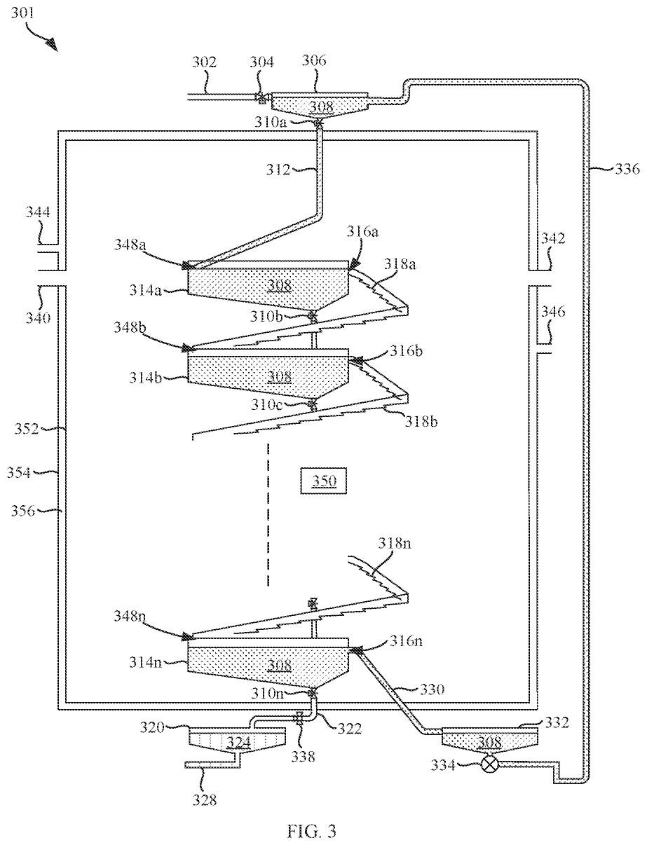

FIG. 3 is a diagram illustrating a bioreactor with plurality of troughs in a single loop providing efficient gas exchange to a cell-culture in the absence of stirring and bubbling to the cell culture with reduced hydrodynamic stress placed upon cells in another embodiment of the present disclosure. The bioreactor (301) of the present disclosure comprises a single loop (336) formed by at least one reservoir (306 and 332), a plurality of open top troughs (314a through 314n) and a plurality of flow channels (318a through 318n) in that the plurality of troughs (314a through 314n) and the plurality of flow channels (318a through 318n) are enclosed within an enclosure say, an inner compartment (352) that is again housed inside an outer compartment (354). The inner and outer compartments (352 and 354) are coupled together in such a way that a hollow passage (356) is created between the outer contour of the inner compartment (352) and the inner contour of the outer compartment (354) that is similar to that of the passage (122) as disclosed in the FIG. 1. The inner compartment (352) comprises a first inlet (340) and a first outlet (342) passing through the outer compartment (354) that enables gas exchange within the inner compartment (352). The outer compartment (354) comprises a second inlet (344) and a second outlet (346) that allows the user to pass through a sterilising gas throughout the passage (356) during the cell cultivation and add a sterilising means, such as a UV sterilisers, throughout the passage (356).

The plurality of open top troughs (314a through 314n) are arranged such that the fresh media and the cell culture flow from the top trough to the bottom trough in a cascade manner, hence referred to herein as ‘cascading troughs’ or a ‘cascade’, and each of the plurality of open top troughs (314a through 314n) comprises an inlet (348a through 348n), an outlet (316a though 316n) and a drain outlet located in the bottom of the trough and more particularly in the nadir with valve (310b through 310n). In an embodiment, the plurality of flow channels (318a through 318n) interconnect the plurality of open top troughs (314a through 314n) by connecting the outlet (316a through 316n) of an upper trough in (348a through 348n) to the inlet (348b through 348n) of the descending trough in (348b through 348n). The plurality of flow channels (318a through 318n) comprises an inclined open top channel with plurality of descending steps either with openings between the steps or with a gas permeable base or a combination of both.

In an embodiment, the reservoirs (306 and 332) and the cultured cell collector may be enclosed within the outer compartment (354) and the inner compartment (352) as required (not shown in the drawing). The reservoir (306) is fed/charged with a cell culture or a cell culture stock (308) externally through an inlet pipe (302) by regulating the levels of the fed cell culture stock (308) within the reservoir (308) by a level control valve (304). The fed cell culture stock (308) from the reservoir (306) is then allowed to flow through a connecting pipe (312) by releasing the drain outlet valve (310a). The cell culture stock (308) thus gets filled in the upper or top most trough (314a) coupled to the connecting pipe (312) wherein the excess amount of cell culture stock gets overflown into the descending trough (314b) through the flow channel (318a). Similarly, all the troughs (314a through 314n) in the cascade get filled with the cell culture stock (308) where the cell cultivation takes place.

In another embodiment, the cell culture stock gets fresh gases containing oxygen that is essential for the growth of cells, is supplied from the first inlet (340) while the waste gases from the culture, such as carbon dioxide, are then removed from the first outlet (342). The internal space (350) of the internal compartment (352) is mostly filled with the fresh gases. The fresh gases can be continuously maintained at a fixed volume in the space (350), using (operating) the valves (not shown in the drawing) in the fresh gas inlet (340) and the outlet (342). The constant volume of the flowing culture and the constant fresh gas in the space (350) can be maintained at a ratio of 1:50. The fresh gas volume can also be increased intermittently if required. Therefore, this method ensures that an appropriate partial pressure difference between the gases contained in the fresh gases and the gases contained in the cell culture are continuously maintained to allow efficient gas exchange.

In another embodiment, a differential or an equilibrium temperature as required, is maintained between the flowing cell culture (308) and the surrounding fresh gas in the space (350). Maintaining a differential temperature when required is possible because the flowing cell culture (308) and the fresh gas are cooled separately and heated externally to the space (350) and their corresponding temperatures are individually controlled. In an example, the continuously circulated cell culture may be quickly cooled or heated (not shown in the drawing) in the loop (336) externally to the space (350) as required. Before the fresh gas enters the inlet (340), it may be quickly cooled or heated (not shown in the drawing) as required. Maintaining an equilibrium temperature when required is possible because of the large surface area interface between the flowing cell culture (308) and the fresh gas which helps to quickly achieve an equilibrium temperature between the flowing cell culture (308) and the fresh gas in the space (350).

Each trough (314a through 314n) may have a shallow depth, which temporarily holds the cell culture (308). A thin stream of cell culture flows in the flow channels (318a through 318n), which creates a gentle agitation to the cell culture (308) in the trough (314a through 314n), therefore assisting in gas exchange. The troughs (314a through 310n) may be rocked (not shown in diagram) if additional agitation is required. In an example, the set of open top troughs (314a through 314n) or a single open top trough may be the set of any troughs with required inlets and outlets or any single trough with required inlets and outlets, for example, a set of fully covered troughs with required inlet and outlets or a fully covered trough with required inlets and outlets.

The flow channels (318a through 318n) comprise plurality of descending steps, which consists of either; openings between the steps, or a gas permeable base, or a combination of both. This, with the help of gravity, will allow diffusion of oxygen (a lighter gas) into the top surface of the flowing cell culture stream and the removal of carbon dioxide (a heavier gas) from the bottom, through these openings/permeable base of the flow channels. Thus, a proper gas exchange takes place in the cell culture within the inner compartment (352). This method of gas exchange avoids the need to mechanically stir or bubble the cell culture, therefore leading to cell cultivation with reduced hydrodynamic stresses placed upon cells.

As the cells grow, they increase in weight and thus settle at the bottom of the troughs (314a through 314n) which may be collected from the drain outlet valves (310b through 310n) of each trough that are coupled to the outlet pipe (322). As shown in FIG. 3, the bioreactor (301) further comprises a cultured cell collector (320) coupled to the drain out valve (310n) of the bottom trough (314n) by means of a metallic or non-metallic tube (322) and a three-way plug valve (338). The cultured cell collector (320) is configured to collect the gravity removed cell culture that will most probably contain more of heavier grown cells (324) from the drain outlet valves of the troughs (314a through 314n) periodically through an outlet pipe (328).

In an embodiment, the overflown cell culture stock (308) that most probably contains lighter cells is collected in the reservoir (332) through a connecting pipe (330) to achieve further growth of the cells via recirculation. This cell culture (308) from the reservoir (332) is re-circulated to the reservoir (306) using anon-impeller pump or a connector pump, for example, that does not place hydrodynamic stress upon cells when it is operational (334). In an embodiment, the three-way valve (338) is configured to divert the cell culture removed from the drain valves (310b through (310n) to the cell culture collector (320). The three-way valve (338) is coupled to the reservoir (320) by a metallic or non-metallic pipe.

In an embodiment, the three-way valve (338) may be configured to divert the cell culture removed from the drain valves (310b through 310n) to the reservoir (322) as required (not shown in drawings). This enables the removed heavier grown cells to be re-circulated back to reservoir (306), in order to increase the overall cell density if required. Once an appropriate cell density is achieved, the three-way valve (338) can be operated to divert the cell culture to the culture cell collector (320), from where it can be harvested.

Alternatively, the heavier grown cells collected in culture cell collector (320) may be re-circulated back to the reservoir (306) either directly by a connector pump (not shown in the drawing) or is sent to an externally located Cell Retention Device (not shown in the drawing) where the cells are separated from the supernatant and re-circulated back to the reservoir (306) in order to increase the cell density if required. Once an appropriate cell density is achieved, the cell culture is harvested from the cultured cell collector (320) and sent to downstream processing. A quantity of the fresh media from an external source which is proportional to the quantity of the harvested cell culture or the quantity of the removed supernatant is fed/charged to the reservoir (306) by opening the valve (304).

In an embodiment, a required quantity of the cell culture may be removed from the culture cell collector (320), discarded and is replaced with the fresh media if required. The removal of the cell culture from drain out valve (310b through 310n) and its re-circulation further contributes to providing a proper gas exchange to the cell culture. Adding sterilising means, such as a UV steriliser or sterile media (or both), in the passage (356) reduces the risk of cross contamination to the cell culture.

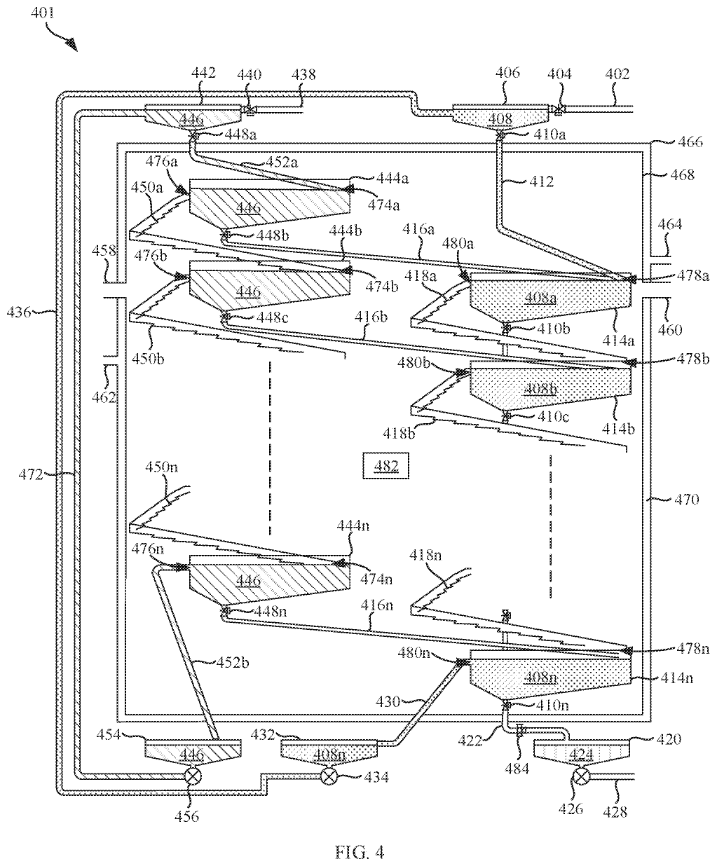

FIG. 4 is a diagram illustrating a bioreactor (401) with plurality of troughs in different loops providing continuous yield in the absence of stirring and bubbling to the cell culture with reduced hydrodynamic stress placed upon cells in yet another embodiment of the present disclosure. The bioreactor (401) of the present disclosure comprises a plurality of open top troughs (414a through 414n and 444a through 444n), a first loop (472), a second loop (436), a plurality of flow channels (416a through 416n; 418a through 418n and 450a through 450n), a cultured cell collector (420), fresh media reservoirs (442 and 454), cell culture reservoirs (406 and 432), liquid level control valves (404 and 440), an outer compartment (466), an inner compartment or an enclosure (468), a first inlet and outlet (458 and 460) for gases, and a second inlet and outlet (462 and 464) to add and remove sterilising gas, such as ozone gas into the passage (470).

The first loop (472) circulates a fresh media (446) throughout the troughs (444a through 444n) from the fresh media reservoirs (442 and 454). Each trough (444a through 444n) comprises an inlet (474a through 474n), an overflow outlet (476a through 476n) and a drain outlet located in the bottom and more particularly in the nadir with a valve (448a through 448n). In one embodiment, a cascade of the plurality of troughs (444a through 444n) are interconnected while the top most (444a) and bottom (444n) troughs are coupled to the fresh media reservoirs (442 and 454) through a connecting pipe (452a and 452b) respectively.

In another embodiment, all the troughs (444a through 444n) are interconnected by a first set of flow channels (450a through 450n) wherein the first set of flow channels (450a through 450n) may be inclined open top channel comprising plurality of descending steps either with openings between the steps or with a gas permeable base or combination of both. In an embodiment, the first set of flow channels (450a through 450n) connects the overflow outlet (476a) of an upper trough (444a) to the inlet (474b) of the descending trough (444b) in the cascade. Further, the valves (448b through 448n) of the drain outlet of the troughs (444a through 444n) are coupled to inlets (478a through 478n) of the troughs (414a through 414n) in the second loop (436) by a second set of flow channels (416a through 416n).

In an example, the fresh media reservoir (442) is positioned at the top of the troughs (444a through 444n) while the fresh media reservoir (454) (also termed as a fresh media collector 454) is placed at, the bottom of the troughs (444a through 444n). The bottom of the fresh media reservoir (454) is coupled to a first connector pump (456) which pumps the overflown contents i.e., fresh media (446) from the troughs (444a through 444n) to the fresh media reservoir (442).

The second loop (436) circulates a stock cell culture (408) throughout the troughs (414a through 414n) from the cell culture reservoirs (406 and 432). Each trough (414a through 414n) comprises an inlet (478a through 478n), an overflow outlet (480a through 480n) and a drain outlet located in the bottom and more particularly in the nadir with a valve (410a through 410n). In one embodiment, a cascade of the plurality of troughs (414a through 414n) are interconnected while the top most (414a) and bottom troughs (414n) are coupled to the cell culture reservoirs (406 and 432) through a connecting pipe (412 and 430) respectively.

In another embodiment, all the troughs (414a through 414n) are interconnected by a third set of flow channels (418a through 418n) wherein the third set of flow channels (418a through 418n) may be inclined open top channel comprising plurality of descending steps either with openings between the steps or with a gas permeable base or combination of both.

In an embodiment, the third set of flow channels (418a through 418n) connects the overflow outlet (480a) of the upper trough (414a) to the inlet (478b) of the descending trough (414b) in the cascade. Further, the drain outlet valves (410b through 410n) are coupled to the outlet pipe (422).

In an example, the cell culture reservoir (406) is positioned at the top of the troughs (414a through 414n) while the cell culture reservoir (432) is placed at the bottom of the troughs (414a through 414n). The bottom of the cell culture reservoir (432) is also coupled to a second connector pump (434) which pumps the overflown contents i.e., cell culture (408n) from the troughs (414a through 414n) to the cell culture reservoir (406).

In another embodiment, the troughs (444a through 444n) in the first loop (472) and the troughs (414a through 414n) in the second loop (436) are interconnected by the second set of flow channels (416a through 416n). The inlet of the second set of flow channels (416a through 416n) is coupled to the drain outlet valves (448b through 448n) of the troughs (444a through 444n) while the outlet is coupled to the inlets (478a through 478n) of the troughs (414a through 414n). The second set of flow channels (416a through 416n) comprise an inclined metallic or non-metallic hollow pass through pipes interconnecting the troughs (444a through 444n) in the first loop (472) to the troughs (414a through 414n) in the second loop (436).

In yet another embodiment, the plurality of open top troughs (444a through 444n and 414a through 414n) along with the plurality of flow channels (416a through 416n; 418a through 418n and 450a through 450n) are enclosed within the inner compartment (468) that is housed inside the outer compartment (466). The inner compartment (468) is housed within the outer compartment (466) in such a way that a hollow passage (470) is left over between inner contour of the outer compartment (466) and outer contour of the inner compartment (468). The inner compartment (468) is provided with the first inlet and outlet (458 and 460) through the outer compartment (466) for circulation or exchange of gases within the bioreactor (401).

In an example, fresh air or gases are pumped into the first inlet (458) which fills the entire space (482) within the inner compartment (468) which is held under a constant pressure between a non-return valve (not shown in the drawing) in the first inlet (458) and a pressure regulating valve (not shown in the drawing) in the first outlet (460). The metabolic waste gases that are released during cell cultivation from the cell culture (408a through 408n) are removed and come out from the first outlet (460). Furthermore, the outer compartment (466) is provided with the second inlet and outlet (462 and 464) for circulation of sterilising gas through the passage (470). The sterilising gas is passed through the second inlet (462) during the cell cultivation process and removed from the second outlet (464). A sterilising means such as an UV steriliser may be added throughout the passage (470). The mechanism or a method that involves in operating the bioreactor (401) of the present disclosure is further described in the following embodiments.

Initially, the fresh media (446) is fed/charged into the fresh media reservoir (442) from an external source through an inlet (438) by regulating the liquid level control valve (440). The level of the fresh media is regulated by operating the liquid level control valve (440). In an embodiment, the trough (444a) is filled with the fresh media (446) through the connecting pipe (452a) by releasing the drain out valve (448a) of the fresh media reservoir (442). As soon as a threshold level of the trough (444a) is reached with excess amount of fresh media, it starts overflowing into the descending trough (444b) through the first set of flow channel (450a). Similarly, all the descendent troughs (444b through 444n) that are in cascade get filled with the fresh media (446) through the first set of flow channels (450b through 450n). Excess amount of fresh media in the bottom trough (444n) is then overflown to the fresh media reservoir (454) which pumps it back to the fresh media reservoir (442) using the first connector pump (456). This fills the entire first loop (472) with the fresh media (446) which provides necessary or essential nutrients and growth promoting components for a cell.

The cell culture (408) as a stock is then fed/charged into the cell culture reservoir (406) externally through an inlet (402) by regulating the liquid level control valve (404). The liquid level control valve (404) controls the level of contents that are present in the cell culture reservoir (406). In an embodiment, the trough (414a) is filled with the cell culture (408) through the connecting pipe (412) by releasing the drain out valve (410a) of the cell culture reservoir (406). Simultaneously, the second set of flow channels (416a) is also released to allow the fresh media (446) from the trough (444a) to flow through to the trough (414a). The cell culture (408) and the fresh media (446) are filled together in the trough (414a) reaching a threshold level with the cell culture (408a). The excess amount of cell culture (408a) is then overflows into the descending trough (414b) through the third set of flow channels (418a).

In an embodiment, the fresh media (446) from the troughs (444a through 444n) is either supplied continuously or in batch wise to the troughs (414a through 414n) as desired. In an embodiment, user may decide the time intervals and amount of the fresh media to be flown to the troughs (414a through 414n) based on which the second set of flow channels (416a through 416n) are operated. Thus, all the descending troughs (414a through 414n) are filled with the cell culture media (408a through 408n) where the cell cultivation takes place.

In yet another embodiment, the drain out valves (410b through 410n) is released at regular intervals to determine the yield and quality of the cultured cells. As the cell cultivation started in the troughs (414a through 414n), the grown cells become heavier and settles down at the bottom of the trough (414a through 414n). Fresh media (446) from the troughs (444a through 444n) may be provided to the culture in the troughs (414a through 414n) at regular intervals in which the lighter cells that are still under culture process in the troughs (414a through 414n) utilize the nutrients and growth promoters from the fresh media (446) as well as gases from the first inlet (458) for culturing.

The grown cells, which are heavier, within the troughs (414a through 414n) are periodically removed from their bottom nadir via the drain out valve (410b through 410n) that are connected to the cultured cell collector (420) through a pipe (422). In one embodiment the heavier grown cells are removed from the bottom nadir of the open top troughs (414a through 414n) before the toxicity develops in the cell culture (408a through 408n) particularly in the bottom of the troughs (414a through 414n) mainly due to the increase in their population and heavier cells, resulting congestion leading to lack of oxygen supply to the cells. Also, the toxicity may develop due to the accumulation of the heavier metabolic waste carbon dioxide at the bottom of the trough resulting from gravity, all of which lead to cell death. The gentle agitation caused in the cell culture (408a through 408n) from the continuous flow of (408) in the second cascade (405) in the absence of stirring and bubbling (408a through 408n) helps heavier cells and the carbon dioxide to sink to the bottom and accumulate more particularly in the bottom nadir of the troughs (414a through 414n).

When the drain out valves (410b through 410n) remain closed or open, the cell culture (408n) continue to overflow into the cell culture reservoir (432) which is pumped to the cell culture reservoir (406) located at the top by using a second pump (434). The cultured cells removed from the drain out valves (410b to 410n) may be diverted via three-way-valve (484) to the reservoir (432) and re-circulated or is diverted to the cultured cell collector (420) to re-circulate or to harvest or to be removed and discarded or sent to a Cell Retention Device as explained under FIG. 3. The quantity of the cell culture or the supernatant removed from the cultured cell collector (420) and out of the bioreactor (401) is replaced by the fresh media in a manner as explained below.

To begin the cell culture process, a required quantity of cell culture (408) is fed/charged to the reservoir (406) from an external source via pipe (402) opening the valve (404). This first quantity of the seed cell culture (408) entering the bioreactor (401) will be the cell culture stock that is required to start the process. Operating the drain out valves (448a and 410a) with drain out valves (448b through 448n and 410b through 410n) result in the appropriate required mixing of the fresh media (446) with the cell culture stock (408) in the beginning of the cell culture process. In an embodiment, the drain out valve (410b through 410n) can be programmed to be opened for 5 seconds and greater than 5 seconds in the frequency range of every 60 seconds and greater than 60 seconds. This removes the heavier grown cells and waste carbon dioxide that have sunk to the bottom of the toughs (414a through 410n), which are eventually re-circulated back to reservoir (406) via connector pump (426).

The lighter cells are overflown in the flow channels (418a through 418n). The gas exchange to the cell culture occurs within the enclosure (482) as explained occurring within the enclosure (350) in FIG. 3 above. The frequent removal of metabolic carbon dioxide rich cell culture and re-circulating the same will remove the built-up metabolic carbon dioxide in each circulation due to the gas exchange occurring in the space (482) for the reasons explained under FIG. 3 above. This method increases the cell viability and takes the culture from a lag phase to log growth phase.

In another embodiment, the drain out valves (448b through 448n and 410b through 410n) can be programmed to open for 5 seconds and greater than 5 seconds in the frequency range of every 60 seconds and greater than 60 seconds. The cultured cell (424) received in the cultured cell collector is then removed out of the bioreactor (401) for harvest and downstream processing. If required, the cell culture that is removed out of the bioreactor is sometimes discarded. If required, the cell culture removed out of the bioreactor (401) is sometimes sent to an external Cell Retention Device where the cells are separated from the supernatant, separated cells are sent back to the reservoir (406), and the supernatant is either discarded or used. This method will help manage a stable stationery cell growth phase with intermittent harvesting of the titre of higher cell quality and higher cell viability for a prolonged period of time.

In an example, the metabolic carbon dioxide rich cell culture can be removed either by gravity or by pumping particularly from the bottom nadir of each or plurality trough (414a through 410 n) or from any other type of container or plurality of container that are used to hold the cell culture, and re-circulating to mix it at the top surface of the cell culture in that container. A skilled person in the related art can apply this method in a top covered trough or similar container thereof. This will assist in the removal of metabolic carbon dioxide from such cell culture in the absence of stirring and bubbling to such cell culture. However, if the cell culture is in such an enclosed container, the space above the surface of that cell culture must be exposed to the continuous supply of the fresh gas which is in contact with the surface of the cell culture. Therefore, an inlet to supply the fresh gas and an outlet to remove gases from such enclosed container is required.

In an embodiment, the operation of the drain out valves (448a through 448n and 410a through 410n) are controlled automatically by a pre-programmed set of instructions through an external device. Furthermore, the time intervals of operating the drain out valves (448a through 448n and 410a through 410n) may vary for different cell cultures.

In an alternative embodiment the drain out valves (448a and 410) and the drain out valves in troughs (444a through 444n and 414a through 414n) are adjusted to remain partially open to allow continuous flow of cultured cells through the bottom drain outlets of the troughs (410b to 410n). In that opening may be adjusted to draw a quantity that is proportional to the rate of growth of a particular cell culture. Furthermore, the opening of the valve may also be adjusted based on the trough's size/capacity. In one embodiment, the overflow (rate) of cell culture is of larger compared to the rate of yield of cell culture.

The space (482) is always filled with a larger proportion of fresh gas and a smaller proportion of metabolic waste gas. In an embodiment, the space (482) is held under greater than the atmospheric pressure for improved diffusion of the fresh gas into both fresh media flowing in the loop (436) and the cell culture flowing in the loop (472). The fresh media from the second set of flow channels (416a through 416n), the cell culture from pipe (412), and the cell culture from the third set of flow channels (418a through 418n) gently flow into and cause gentle agitation to the cell culture (408a through 408n) that is shallow in depth. The ratio between the height and the surface perimeter measurements of the cell culture in each trough (444a through 444n and 414a through 414n) can be kept at 1:20 which may be adjusted. The ratio between the height and the surface perimeter measurements of the cell culture flowing in each flow channel (450a through 450n and 418a through 418n) can be kept at 1:1000 which may be adjusted. Thus, adequate homogeneity to distribute the nutrients is maintained in the shallow cell culture (408a through 408n) contained in the troughs (414a through 414n) in the absence of stirring and bubbling actions.

For similar reasons, the fresh media is maintained oxygen rich. In addition to the gas exchange and agitation mechanisms described in FIG. 3, the frequent addition of oxygen rich fresh media from second set of flow channels (416a through 416n) to the shallow cell culture (408a through 408n) in the troughs (414a through 414n) also gives adequate nutrient's supply, maintain adequate homogeneity, and efficient gas exchange to the shallow cell culture (408a through 408n) in the troughs (414a through 414n). The troughs (444a through 444n) and more particularly the troughs (414a through 410n) may be rocked if additional agitation is required.

In an example, the set of open top trough (444a through 444n and 414a through 414n) or a single open top trough may be the set of any troughs with required inlets and outlets or any single trough with required inlets and outlets, for example, a set of fully covered troughs with required inlets and outlets or a fully covered trough with required inlets and outlets.

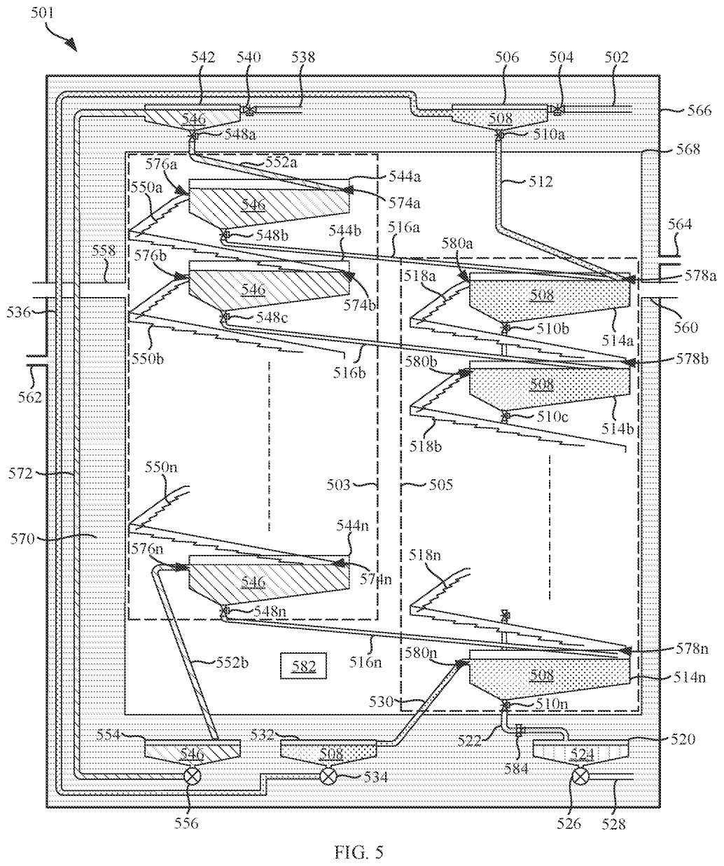

FIG. 5 is a diagram illustrating a bioreactor (501) comprising at least two cascades accelerating the yield of cells in the absence of stirring and bubbling to the cell culture with reduced hydrodynamic stress placed upon cells in yet another additional embodiment of the present disclosure. As shown there, the bioreactor (501) comprises a first cascade (503), a second cascade (505), a set of interconnecting flow channels (516a through 516n), a first set of reservoirs (542 and 554), a second set of reservoirs (506 and 532) and a collector (520) that are housed inside a first enclosure (566).

The first cascade (503) comprises a first set of open top troughs (544a through. 544n) that are interconnected by a first set of flow channels (550a through 550n) wherein the first cascade (503) circulates a fresh media (546) throughout the first set of open top troughs (544a through 544n). The second cascade (505) comprise a second set of open top troughs (514a through 514n) that are interconnected by a second set of flow channels (518a through 518n) wherein the second cascade (505) circulates a cell culture (508) throughout the second set of open top troughs (514a through 514n).

In an embodiment, each of the first and second set of open top troughs (544a through 544n and 514a through 514n) comprises an inlet (574a through 574n and 578a through 578n), an outlet (576a through 576n and 580a through 580n) and drain outlets (548a through 548n and 510a through 510n). The inlets (574a through 574n and 578a through 578n) and outlets (576a through 576n and 580a through 580n) of the open top troughs may be provided with a multi-valve plug that enables them to couple with the plurality of interconnecting flow channels as per the requirement. The first set of flow channels (550a through 550n) interconnects the outlet (576a through 576n) of a leading trough (544a through 544n-1) to the inlet (574b through 574n) of a descending trough (544b through 544n) in the first cascade (503).

Similarly, the second set of flow channels (518a through 518n) interconnects the outlet (580a through 580n) of a leading trough (514a through 514n) to the inlet (578b through 578n) of a descending trough (514b through 514n) in the second cascade (505). In an embodiment, the first and second set of flow channels (550a through 550n and 518a through 518n) may be inclined open top channels comprising plurality of descending steps either with openings between the steps or with a gas permeable base or a combination of both.

The first set of reservoirs (542 and 554) comprises a closed container provided with an inlet, outlet and, a drain outlet which carries the fresh media (546). In an embodiment, the reservoir (542) is fed with the fresh media (546) through an inlet pipe (538) coupled with a liquid level control valve (540). The liquid level control valve (540) helps to maintain a threshold or desired levels of the fresh media (546) within the reservoir (542). In another embodiment, the reservoir (554) is positioned below the reservoir (542) which collects the fresh media (546) from the first cascade (503). Further, the drain outlet of the reservoir (554) is connected to an external outlet pipe (570) that connects between the reservoir (554) and the reservoir (542) through a connector pump (556). The fresh media (546) collected in the reservoir (554) is re-circulated back to the reservoir (542).

The second set of reservoirs (506 and 532) comprises a closed container provided with an inlet, outlet and a drain outlet which carries the cell culture (508). In an embodiment, the reservoir (506) is initially fed with a starting seed cell culture stock (not shown in the drawing) through an inlet pipe (502) coupled with a liquid level control valve (504). The liquid level control valve (504) helps to maintain a threshold or desired levels of the cell culture (508) within the reservoir (506). In another embodiment, the reservoir (532) is positioned below the reservoir (506) which collects the cell culture (508) from the second cascade (505). Further, the drain outlet of the reservoir (532) is connected to an external outlet pipe (536) that connects between the reservoir (532) and (506) through a connector pump (534). The cell culture (508) collected in the reservoir (532) is re-circulated back to the reservoir (508). The plurality of interconnecting flow channels (516a through 516n) comprises a closed or open top channel that is inclined to couple a plurality of drain outlet valves (548b through 548n) of the open top troughs (544a through 544n) in the first cascade (503) to the inlets (578a through 578n) of the open top troughs (514a through 514n) in the second cascade (505). This plurality of interconnecting flow channels (516a through 516n) thus facilitates the flow of the fresh media (546) from the first cascade (503) into the second cascade (505) where the cell culture (508) utilises all nutrients and growth promoting components in the fresh media (546) for cell cultivation.

In an embodiment, the cultured cell collector 520) comprises a closed container with an inlet pipe (522) coupled with a three-way or multi-valve plug (584) and an outlet coupled to an external outlet pipe (528) through a connector pump (526). A pipe (not shown in the drawing) connects between the three-way or multi-valve plug (584) and the reservoir (532). The inlet pipe (522) of the culture cell collector (520) is coupled to the drain outlets of the open top troughs (514a through 514n) with valve (510b through 510n) in the second cascade (505). In an embodiment, the first and second cascades (503 and 505) along with the plurality of interconnecting flow channels (516a through 516n) are enclosed within a second enclosure (568) provided with an inlet (558) and an outlet (560) passing through the first enclosure (566). The inlet (558) and outlet (560) enables gas exchange within the bioreactor (501) in that the inlet (558) allows the fresh gases to pass through and fills entire space (582) of the second enclosure (568) while the outlet (560) releases the waste gases obtained during cell cultivation into the atmosphere. In an example, the space (582) of the second enclosure (568) is always filled with a fresh gas providing necessary aeration for growth of cells.

The first enclosure (566) also comprises an inlet (562) and an outlet (564) facilitating the circulation of a sterilising gas in the passage (570). The passage (570) may comprise sterilising gas such as ozone gas or ultra-violet rays or both that helps in eliminating cross contamination between outside and inside the bioreactor (501) during cell cultivation. The sterilising medium in the passage (570) also keeps the exterior of the first and second set of reservoirs (542, 554 and 506, 532), the culture cell collector (520) and inlets and/or outlets coupled to at least one of the first cascade (503), second cascade (505), and the external outlet pipes sterile during the cell culture. This arrangement between the first and second enclosures (566 and 568) overall helps in reducing or eliminating cross contamination during the cell cultivation process.

As the cell culture (508) in the second cascade (505) utilises the flow of the oxygen rich fresh media (546) from the first cascade (503), the cells grow quicker with efficient gas exchange in the second enclosure (568) which makes them to settle down in the open top troughs (514a through 514n) due to increase in their mass and gravity. Then the heavier grown cells with the heavier metabolic carbon dioxide in the cell culture is re-circulated to remove the metabolic carbon dioxide and diffuse oxygen into the cell culture as explained in FIG. 4 above. The troughs (544a through 544n) and more particularly the troughs (514a through 510n) may be rocked if additional agitation is required.

Cell culture (524) quantity in the bioreactor (501) reduces when it is removed from the bioreactor (501) through the pipe (528) for harvest and downstream processing. It may also be transported from the bioreactor (501) to a Cell Retention Device, where the supernatant is separated from cells and discarded. Then the separated cells are sent back to the bioreactor (501) via the reservoir (506). If needed, the cell culture can be removed from the bioreactor (501) and discarded. The quantity of the removed cell culture from the second cascade (505) and out of the bioreactor (501) is proportional to the fresh media (546) that is added into the bioreactor (501) from an external source into the first cascade (503) via reservoir (542).

The following methods provides efficient gas exchange to a flowing cell culture, in the absence of stirring and bubbling, and reducing hydrodynamic stresses upon cells. This method of gas exchange occurs within a space (582) protected by a sterile envelope or passage (570) and further protected by the outer body of the first enclosure (566). The space (582) is maintained at a fixed constant volume of fresh gas that can be intermittently increased if required. There is a large surface area interface between the cell culture (508) and the surrounding fresh gas in the space (582). There is continuous removal of metabolic carbon dioxide (that builds up in the cell culture) and diffusion of the oxygen into the cell culture (508) in each circulation of the cell culture (508) within the space (582).

The ability of the bioreactor (501) to induce quick temperature changes when required to the flowing cell culture (508) and the fresh gas in the space (582) will assist better temperature control of the culture. The cell culture and the fresh gas can be maintained in equilibrium and differential temperature conditions in the space (582). This can be adjusted if required thus proving beneficial for studying the cell behaviour and characteristics. In an example, sterile access to the cell culture (508) in each trough (514a through 514n) from the bioreactor (501) exterior is provided (not shown in the drawing) to contact cell culture with biological substance/s. In another example, sterile access to the cell culture (508) in each trough (514a through 514n) from the bioreactor (501) exterior is provided to introduce or remove biological substances, such as tissues or organoids, from each trough (514a through 514n).

In an alternative embodiment (not shown in the drawing), the open top troughs (514a through 514n) may be implemented as a storage container/container, for example plastic bag or a bag made from other materials thereof, referred to herein as the ‘cell bag’ that are disposable. The cell bag is partly filled with cell culture (508), wherein the fresh gases are injected into the cell bags and removed from it. The cell culture is removed from the bottom nadir of the cell bag. The culture is received into the cell bag from the first cascade (503) via interconnecting flow channels or tubes (516a through 516n), wherein the second cascade (505) continuously circulates the cell culture (508). Although the continuous flow of (508) in and out of the cell bag gently agitates the cell culture (508) contained within the cell bag, a rocking or some other means external to the cell bag can impart additional agitation to the cell culture (508) contained in the cell bag.

In an example, the wall of each cell bag that is Placed in each open top trough (514a through 514n) may be permeable. In an example, the interconnecting flow channel or tubes (516a through 516n) may be permeable. In another example, the flow channels (518a through 518n) may be permeable tubes. In yet another example (not shown in the drawing), each cell bag will have an inlet for fresh gas supply and outlet to remove gases from the cell bag, and both such inlet and outlet are coupled to plastic tubes that deliver the fresh gas from a source located external to the bioreactor (501) into the cell bags and exhaust the gas from the cell bags to outside the bioreactor (501).

The arrangements in the examples above can also be applied to circulate the fresh media (546) in the cascade (503). In another example, the method of continuous process having the potential to deliver all or some benefits from the application of the present disclosure is also made possible in a single cell bag or in a single open top trough or in a single cell bag placed inside an open top trough (544a through 544n and 514a through 514n).

In another example, the method of continuous process having the potential to deliver all or some benefits from the application of the present disclosure is also made possible in a single open top trough or in a single enclosed trough with the required inlets and outlets, or a set of enclosed troughs with the required inlets and outlets as in (544a through 544n and 514a through 514n).

While various embodiments of the present disclosure have been described above, it should be understood that they have been presented by way of example only, and not limitation. Thus, the breadth and scope of the present disclosure should not be limited by any of the above-discussed embodiments but should be defined only in accordance with the following claims and their equivalents.

1. A bioreactor for cell culture comprising:
a first set of open top troughs that are interconnected by a first set of flow channels to form a first cascade;
a second set of open top troughs that are interconnected by a second set of flow channels to form a second cascade; and
a set of interconnecting flow channels coupled between the first cascade and the second cascade,
wherein the first cascade circulates a fresh media and the second cascade circulates a cell culture and a first quantity of the fresh media from first cascade is supplied to the second cascade through the set of interconnecting flow channels when a second quantity of cell culture is removed from the second cascade.

2. The bioreactor of claim 1, wherein the first and the second set of flow channels are inclined open top channels comprising at least one of a plurality of descending steps with openings between the steps and a gas permeable base.

3. The bioreactor of claim 1, wherein the set of interconnecting flow channels are inclined and closed hollow tubes allowing a free flow of the fresh media from the first cascade to the second cascade.

4. The bioreactor of claim 1, wherein the first set of flow channels are correspondingly coupled between a first set of overflow outlets in the first set of open top troughs and a first set of inlets in the next of the first set of open top troughs to form the first cascade.

5. The bioreactor of claim 4, wherein the second set of flow channels are correspondingly coupled between a second set of overflow outlets in the second set of open top troughs and a second set of inlets in the next of the second set of open top troughs to form the second cascade.

6. The bioreactor of claim 5, wherein the first cascade, the second cascade and the set of interconnecting flow channels are enclosed within a second enclosure that is housed inside a first enclosure.

7. The bioreactor of claim 6, wherein the fresh media is fed to the first cascade by a one or more reservoir and the cell culture is fed to the second cascade by a one or more reservoir.

8. The bioreactor as claimed in claim 7, wherein the second enclosure is supplied with a fresh gas facilitating a gas exchange for the cell culture during cell cultivation and to maintain a constant volume of the fresh gas which is increased intermittently.

9. The bioreactor as claimed in claim 8, wherein the first enclosure is infused with a one or more sterilizing means to eliminate cross contamination during the cell cultivation.

10. The bioreactor as claimed in claim 1 wherein the first and the second set of troughs comprises a set of cell bags each having a nadir at the bottom, that are partly filled with cell culture wherein the cell culture at the bottom is removed through the nadirs, in that, the set of troughs comprising at least one trough and the set of cell bags comprising at least one cell bag.

11. A bioreactor for cell culture comprising at least one trough participating to form a cascade, the cascade housed within a first enclosure containing a fresh gas, the cascade operative to circulate a cell culture to interact with the fresh gas, wherein said at least one trough comprising an overflow outlet at the top to circulate said cell culture through said cascade and an outlet at the bottom coupled to a valve for collecting grown cell culture that settle down due to gravity.

12. The bioreactor of claim 11,
further comprising a set of troughs participating to form the cascade wherein each trough in the set of trough having the overflow outlet at the top to circulate said cell culture through said cascade.

13. (canceled)

14. The bioreactor of claim 12, further comprising a set of flow channels coupled between the overflow outlet of one trough to an inlet of another trough that is next in the set of troughs to form the cascade.

15. The bioreactor of claim 14, wherein the first enclosure is housed within a second enclosure and the space between the first enclosure and the second enclosure is infused with a sterile medium to eliminate cross contamination during the cell cultivation.

16. A method of cultivation of cell culture in a bioreactor comprising:
cascading a fresh medium through a first set of troughs;
cascading a cell culture through a second set of troughs;
allowing a grown cell culture to settle down due to gravity at the bottom of each of the second set of troughs, while cascading a remaining cell culture through the second set of troughs; and
collecting a first quantity of the grown cell culture from the bottom of the second set of troughs;

17. The method of claim 16, further comprising replenishing a second quantity of the fresh media from the first set of troughs to the second set of trough, in that the first quantity of the grown cell culture is proportional to the second quantity of the fresh media.

18. The method of claim 17, wherein the collecting the first quantity of the grown cell culture and the replenishing of second quantity of the fresh media is continuous, in that, the second quantity of the fresh media is substantially lesser than the remaining cell culture in the bioreactor.

19. The method of claim 17, wherein the collecting the first quantity of the grown cell culture and the receiving of the second quantity replenishing fresh media into the bioreactor is intermittent.

20. The method of claim 16, further comprising charging the first cascade with the fresh medium and charging the second cascade with a fresh cell culture when the grown cell culture is collected.

21. The method of claim 20, further comprising charging the second cascade in part with the grown cell culture collected.

22. The method of claim 16, wherein the cascading comprises at least one of allowing a free flow of the fresh medium and the cell culture from one trough to another, rocking the first and second set of troughs and guiding through channels the fresh medium and the cell culture from one trough to another.

23. The method of claim 16, further comprising maintaining a fresh gas at a first temperature and maintaining the cell culture at a second temperature within the bioreactor.

24. A method of growing a cell culture comprising cascading the cell culture through at least one trough housed within a first enclosure containing a fresh gas, the cascading enabling the cell culture to interact with the fresh gas.

25. The method of claim 24, further comprising cascading the cell culture through a set of troughs that are open at the top.

26. The method of claim 25, further comprising collecting grown cell culture that settle down in each trough in the set of troughs through a set of outlets at the bottom and a set of valves coupled to the set of outlets.

27. The method of claim 26, further comprising cascading the cell culture through a set of flow channels coupled between a set of overflow outlets in the set of troughs to a set of inlets in the next of the set of troughs.

28. The method of claim 28, further comprising preventing cross contamination of cell culture by infusing a sterile medium into a space between the first enclosure and a second enclosure, wherein the first enclosure is within the second enclosure.
